(19) United States
(12) Patent Application Publication
(10) Pub. No.: US 2010/0234347 A1
(43) Pub. Date: Sep. 16, 2010

(54) Substituted Pteridines substituted with a Four-Membered Heterocycle

(75) Inventors: Horst Dollinger, Schemmerhofen (DE); Domnic Martyres, Biberach (DE); Rolf Goeggel, Ulm (DE); Birgit Jung, Laupheim (DE); Peter Nickolaus, Warthausen (DE)

Correspondence Address: MICHAEL P. MORRIS;BOEHRINGER INGELHEIM USA CORPORATION, 900 RIDGEBURY RD, P O BOX 368, RIDGEFIELD, CT, 06877-0368, US

(73) Assignee: Boehringer Ingelheim Internationl GmbH, Ingelheim (DE)

(21) Appl. No.: 12/301,861

(22) PCT Filed: May 15, 2007
(86) PCT No.: PCT/EP2007/054709

§ 371 Date: May 27, 2010

(30) Foreign Application Priority Data
May 24, 2006 (EP) 06114539.7

(51) Int. Cl. A61K 31/397; C07D 475/08; A61P 25/28; A61P 25/24; A61P 25/22; A61P 25/18; A61P 25/16; A61P 35/00; A61P 35/02

(52) U.S. Cl. 514/210.21; 544/260

(57) ABSTRACT

The invention relates to novel pteridines of formula (1),

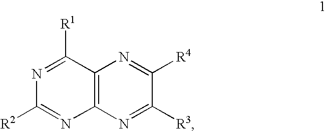

suitable for the treatment of airway or gastrointestinal complaints or diseases, inflammatory diseases of the joints, the skin or eyes, diseases of the peripheral or central nervous systems or cancerous diseases and pharmaceutical compositions comprising said compounds.

The invention relates to new pteridines which are suitable for treating
respiratory or gastrointestinal complaints or diseases,
inflammatory diseases of the joints, skin or eyes,
diseases of the peripheral or central nervous system or
cancers,
   as well as pharmaceutical compositions which contain these compounds.

### PRIOR ART

Pteridines are known from the prior art as active substances with an antiproliferative activity. Merz et al. describe in the Journal of Medicinal Chemistry 1998, 41, 4733-4743 the preparation of 7-benzylamino-6-chloro-2-piperazino-4-pyrrolidinopteridine and derivatives thereof which are free from positional isomers. It has been shown that the compounds prepared are able to inhibit the growth of tumour cells. DE 3540952 describes 2-piperazino-pteridines which are substituted in the 6-position by a halogen atom selected from among a fluorine, chlorine or bromine atom. It has been shown that these compounds were able to inhibit the activity of tumour cells and human thrombocytes in vitro. DE 3323932 discloses 2-piperazino-pteridines which carry a dialkylamino, piperidino, morpholino, thiomorpholino or 1-oxidothiomorpholino group in the 4-position. It has been shown that these compounds were able to inhibit the activity of tumour cells and human thrombocytes in vitro. DE 3445298 describes pteridines with a large number of different substituents in the 2, 4, 6 and 7-position, while compounds with a 2-piperazino group on the pteridine skeleton are suitable as inhibitors of tumour growth and also have antithrombotic and metastasis-inhibiting properties. U.S. Pat. No. 2,940,972 discloses tri- and tetrasubstituted pteridine derivatives, commenting in general terms that these pteridines have valuable pharmacological properties, namely coronary artery dilating, sedative, antipyretic and analgesic activities.

The phosphodiesterase 4 inhibitors known from the prior art are known to trigger side effects such as nausea and vomiting (Doherty, 1999, Curr. Op. Chem. Biol., August 3, (4):466-73). The substances mentioned in this invention preferably inhibit the B-isoenzymes of phosphodiesterase 4, are therefore preferred PDE4B-inhibitors and are consequently particularly suitable for treating the above-mentioned diseases, as, unlike other PDE4-inhibitors which preferably inhibit the other PDE4-isoenzymes (e.g. isoenzymes A, C or D), they do not trigger these side effects in an animal model for nausea and vomiting (S. Murinus, Yamamoto K. et al., Physiol. Behay., 2004, Oct. 30, 83(1), 151-6).

The aim of the present invention is to provide new compounds which are suitable for the prevention or treatment of respiratory or gastrointestinal complaints or diseases, inflammatory diseases of the joints, skin or eyes, diseases of the peripheral or central nervous system, or cancers, particularly those compounds which are characterised by reduced side effects, particularly emesis and nausea.

### DESCRIPTION OF THE INVENTION

Surprisingly it has now been found that pteridines of formula 1 are suitable for treating inflammatory diseases.

The present invention therefore relates to compounds of formula 1

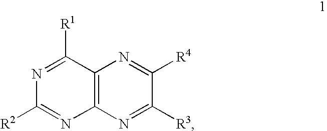

wherein
R¹ denotes a saturated or unsaturated, four-membered heterocyclic group, which contains a nitrogen atom and may optionally contain a further atom selected from among nitrogen, sulphur and oxygen;
R² denotes halogen, OR^{2.1}, SR^{2.1}, NR^{2.1}R^{2.2},
where
   R^{2.1} denotes H, C₆₋₁₀-aryl, C₇₋₁₁-aralkyl;
   R^{2.2} denotes H, C₆₋₁₀-aryl, C₇₋₁₁-aralkyl;
or
R² denotes a group selected from among C₆₋₁₀-aryl, C₅₋₁₀-heteroaryl and a five-, six- or seven-membered heterocyclic group, which contains a nitrogen atom and which may optionally contain a further atom selected from among nitrogen, sulphur and oxygen, while this group may optionally be substituted by a group selected from among C₁₋₆-alkyl, C₃₋₆-cycloalkyl, C₇₋₁₁-aralkyl and N(C₁₋₄-alkyl)₂
R³ denotes a group of formula 1a,

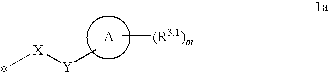

wherein
A denotes a ring selected from among a monocyclic, heterocyclic ring, a bicyclic ring which optionally contains one or more heteroatoms, a C₆₋₁₀-aryl and a C₅₋₁₀-heteroaryl,
X denotes NR^{3. 2}, O, S;
Y denotes C₁₋₄-alkylene, which may optionally be substituted by one or more R^{3.3},
m denotes 0, 1, 2 or 3
R^{3.1} each independently of one another denote C₁₋₆-alkyl, C₆₋₁₀-aryl, COOR^{3.1.1}, CONR^{3.1.1}R^{3.1.2}, CN, NR^{3.1.1}R^{3.1.2}, NHCOR^{3.1.1}, OR^{3.1.1}, O—C₁₋₆-haloalkyl, SO₂R^{3.1.1}, SO₂NH₂, halogen, C₁₋₆-haloalkyl, C₁₋₆-alkyl-CONR^{3.1.1}R^{2.1.2}, C₁₋₆-alkyl-NR^{3.1.1}R^{3.1.2}, C₁₋₆-alkyl-CONH₂, O—C₁₋₆-alkylene-NH₂, O—C₃₋₆-cycloalkyl, O—C₁₋₄-alkylene-C₃₋₆-cycloalkyl, O—C₁₋₄-alkylene-CONH₂, SO₂NR^{3.1.1}R^{3.1.2};
   or
R^{3.1} together with two atoms of A forms a 5- or 6-membered carbocyclic ring or a 5- or 6-membered heterocyclic ring which may optionally contain one or more heteroatoms selected from among oxygen and nitrogen,
wherein
   R^{3.1.1} denotes H, C₁₋₆-alkyl;
   R^{3.1.2} denotes H, C₁₋₆-alkyl;
and
R^{3.2} denotes H, C₁₋₆-alkyl;
and wherein
R^{3.3} each independently of one another denote H, C₁₋₆-alkyl, C₁₋₆-alkyl-OH, C₃₋₆-cycloalkyl, C₃₋₆-cycloalkyl-OH, O—C₁₋₆-alkyl, COOR^{3.1.1}, COO—C₁₋₆-alkyl, CONR^{3.1.1}R^{3.1.2};
or
R^{3.3} together with one or two carbon atoms of Y forms a carbocyclic ring with 3, 4, 5 or 6 carbon atoms,
R⁴ denotes a group selected from among halogen, C₁₋₆-alkyl, C₂₋₆-alkenyl, C₂₋₆-alkynyl, C₃₋₆-cycloalkyl, C₃₋₆-cycloalkenyl, OR^{4.1}, SR^{4.1}, C₁₋₆-haloalkyl, NR^{4.1}R^{4.2} or a group selected from among C₆₋₁₀-aryl, 3-10-membered heterocyclic group and C₅₋₁₀-heteroaryl, which may optionally be substituted by one or more groups selected from among C₁₋₆-alkyl, C₁₋₆-haloalkyl, CN, O—C₁₋₆-alkyl, halogen,
   R^{4.1} denotes H, C₁₋₆-alkyl, C₆₋₁₀-aryl, C₇₋₁₁-aralkyl;
   R^{4.2} denotes H, C₁₋₆-alkyl, C₆₋₁₀-aryl, C₇₋₁₁-aralkyl;
and pharmacologically acceptable salts, diastereomers, enantiomers, racemates, hydrates or solvates thereof.

In another aspect the invention relates to the above mentioned compounds of formula 1, wherein R², R³ and R⁴ have the above definitions and wherein
R¹ denotes an azetidine ring
and pharmacologically acceptable salts, diastereomers, enantiomers, racemates, hydrates or solvates thereof.

In another aspect the invention relates to the above mentioned compounds of formula 1, wherein R³ and R⁴ have the above definitions and wherein
R¹ denotes an azetidine ring,
R² denotes a five- or six-membered heterocyclic group which contains one or two nitrogen atoms;
and pharmacologically acceptable salts, diastereomers, enantiomers, racemates, hydrates or solvates thereof.

In another aspect the invention relates to the above mentioned compounds of formula 1, wherein R³ and R⁴ have the above definitions and wherein
R¹ denotes an azetidine ring;
R² denotes a six-membered heterocyclic group which contains two nitrogen atoms;
   and pharmacologically acceptable salts, diastereomers, enantiomers, racemates, hydrates or solvates thereof.

Also preferred are the above mentioned compounds of formula 1, wherein R¹, R² and R³ have the above definitions and wherein
R⁴ denotes a group selected from among Cl, F, Br, methyl, ethyl, propyl, 2-methylpropyl, cyclopropyl, cyclohexyl, methoxy, CF₃, NR^{4.1}R^{4.2},
   C₆₋₁₀-aryl, optionally substituted by one or more groups selected from among methyl, CF₃, CN, methoxy, fluorine, chlorine,
   a five- or six-membered heterocyclic ring, which may contain one or more heteroatoms selected from among nitrogen and oxygen,
   a five-membered heterocyclic aromatic group, which may contain one or more groups selected from among nitrogen and oxygen,
   an aromatic or non-aromatic bicyclic group, which may contain one or more heteroatoms selected from among sulphur and oxygen contain may;
   R^{4.1} denotes H, methyl, ethyl;
   R^{4.2} denotes methyl, ethyl, phenyl;
and pharmacologically acceptable salts, diastereomers, enantiomers, racemates, hydrates or solvates thereof.

Particularly preferred are the above mentioned compounds of formula 1, wherein R¹, R² and R³ have the above definitions and wherein
R⁴ denotes Cl,
   as well as pharmacologically acceptable salts, diastereomers, enantiomers, racemates, hydrates or solvates thereof.

In another preferred aspect the invention relates to the above compounds of formula 1, wherein R¹, R² and R⁴ have the above definitions and wherein
R³ denotes a group of general formula 1a, wherein
   A denotes a five-, six- or seven-membered heterocyclic, aromatic or non-aromatic ring, or
      an aromatic or non-aromatic, bicyclic ring consisting of eight, nine or ten atoms, which optionally contains one, two or three heteroatoms;
   X denotes NR^{3.2}, O, S;
   Y denotes C₁₋₄-alkylene, which may optionally be substituted by one or more R^{3.3}
   m denotes 0, 1, 2 or 3;
   R^{3.1} each independently of one another denote C₁₋₄-alkyl, C₆₋₁₀-aryl, COOR^{3.1.1}, CONTR^{3.1.1}R^{3.1.2}, CN, NR^{3.1.1}R^{3.1.2}, NHCOR^{3.1.1}, OR^{3.1.1}, O—C₁₋₄-haloalkyl, SO₂R^{3.1.1}, SO₂NH₂, halogen;
      R^{3.1.1} denotes H, C₁₋₆-alkyl;
      R^{3.1.2} denotes H, C₁₋₆-alkyl;
   R^{3.2} denotes H, C₁₋₆-alkyl;
   R^{3.3} each independently of one another denote C₁₋₆-alkyl, C₁₋₆-alkyl-OH, C₃₋₆-cycloalkyl, O—C₁₋₆-alkyl, COOH, COO—C₁₋₆-alkyl, CONH₂;
   R^{3.3} together with one or two carbon atoms of Y forms a carbocyclic ring with 3, 5 or 6 carbon atoms
and pharmacologically acceptable salts, diastereomers, enantiomers, racemates, hydrates or solvates thereof.

In another preferred aspect the invention relates to the above compounds of formula 1, wherein R¹, R² and R⁴ have the above definitions and wherein
R³ denotes a group of general formula 1a, wherein
   A denotes a five-, six- or seven-membered heterocyclic, aromatic or non-aromatic ring, which contains one, two or three heteroatoms, independently of one another, selected from among oxygen, nitrogen and sulphur; or
      an aromatic or non-aromatic bicyclic ring consisting of eight, nine or ten atoms, which optionally contains one, two or three heteroatoms, independently of one another, selected from among oxygen, nitrogen and sulphur;
   X denotes NR^{3.2}, O, S;
   Y denotes C₁₋₂-alkylene, which may optionally be substituted by one or more R^{3.3}
   m denotes 0, 1, 2 or 3;
   R^{3.1} each independently of one another denote C₁₋₄-alkyl, C₆₋₁₀-aryl, COOH, COO—C₁₋₄-alkyl, CONH₂, CN, NH₂, NHCO—C₁₋₄-alkyl, OH, O—C₁₋₄-haloalkyl, SO₂—C₁₋₄-alkyl, SO₂NH₂ or halogen;
   R^{3.2} denotes H, or C₁₋₄-alkyl;
   R^{3.3} denotes H, C₁₋₄-alkyl-OH, C₃₋₆-cycloalkyl, COOH, COO—C₁₋₄-alkyl or CONH₂,
   or wherein
   R^{3.3} together with one or two carbon atoms of Y forms a carbocyclic ring with 3, 5 or 6 carbon atoms,
as well as pharmacologically acceptable salts, diastereomers, enantiomers, racemates, hydrates or solvates thereof.

In another preferred aspect the invention relates to the above compounds of formula 1, wherein R¹, R² and R⁴ have the above definitions and wherein
R^{3.1} each independently of one another denote methyl, ethyl, propyl, Ph, COOH, COOMe, CONH₂, CN, NH₂, NHCOMe, OH, OMe, OEt, OCF₃, OCHF₂, SO₂Me, SO₂NH₂, F, Cl, Br;
R^{3.2} denotes H, C₁₋₄-alkyl;
R^{3.3} denotes H, methyl, ethyl, propyl, butyl, CH₂OH, CH₂CH₂OH, C(CH₂)₂OH, cyclopropyl, COOH, COOMe, COOEt, COOPr, CONH₂, OMe, OEt, OPr
or wherein
R^{3.3} together with one or two carbon atoms of Y forms a carbocyclic ring with 3, 5 or 6 carbon atoms,
as well as pharmacologically acceptable salts, diastereomers, enantiomers, racemates, hydrates or solvates thereof.

In another preferred aspect the invention relates to the above compounds of formula 1, wherein R¹, R², R³ and R⁴ have the above definitions and wherein
R^{3.1} denotes methyl, iso-propyl, tert-butyl;
R^{3.2} denotes H, methyl;
R^{3.3} denotes H, methyl;
and pharmacologically acceptable salts, diastereomers, enantiomers, racemates, hydrates or solvates thereof.

Also particularly preferred are compounds of formula 1, wherein
R³ denotes a group of general formula 1a, wherein
   A denotes a saturated or unsaturated, mono- or bicyclic C₅₋₁₀ heterocycle with 1, 2 or 3 heteroatoms selected from among N, O and S;
   X denotes NR^{3.2}, O;
   Y denotes C₁₋₂-alkylene, optionally substituted by one or more R^{3.3}
   m denotes 0, 1, 2 or 3;
   R^{3.1} denotes methyl,
   R^{3.2} denotes H,
   R^{3.3} denotes H,
and pharmacologically acceptable salts, diastereomers, enantiomers, racemates, hydrates or solvates thereof.

In another preferred aspect the invention relates to the above compounds of formula 1, wherein R¹, R² and R⁴ have the above definitions and wherein
R³ denotes a group of general formula 1a, wherein
   A denotes thiophene, furan, pyrazole, pyridine, isoxazole, thiazole, benzimidazole, benzo[b]thiophene, 2,3-dihydrobenzodioxin, 1,2,3,4-tetrahydronaphthaline, oxazole, tetrahydrofuran or tetrahydropyran;
   X denotes NR^{3.2}, O;
   Y denotes C₁₋₂-alkylene, optionally substituted by one or more R^{3.3}
   m denotes 0, 1, 2 or 3;
   R^{3.1} denotes methyl,
   R^{3.2} denotes H,
   R^{3.3} denotes H,
and pharmacologically acceptable salts, diastereomers, enantiomers, racemates, hydrates or solvates thereof.

In another preferred aspect the invention relates to compounds of formula 1, wherein
R¹ denotes azetidine
R² denotes piperazine
R³ denotes a group of general formula 1a, wherein
   A denotes thiophene, furan, pyrazole, pyridine, isoxazole, thiazole, benzimidazole, benzo[b]thiophene, 2,3-dihydrobenzo[1,4]dioxin, 1,2,3,4-tetrahydronaphthaline, oxazole or phenyl, tetrahydrofuran or tetrahydropyran;
   X denotes NR^{3.2}, O;
   Y denotes methylene or ethylene
   m denotes 0, 1, 2 or 3;
   R^{3.1} each independently of one another denote methyl, ethyl, phenyl, halogen, COOR^{3.1.1}, CONR^{3.1.1}R^{3.1.2}, CN, NR^{3.1.1}R^{3.1.2}, NCHOR^{3.1.1}, OR^{3.1.1}, O—C₁₋₃-haloalkyl, SO₂R^{3.1.1}, SO₂NH₂, C₁₋₃-haloalkyl, wherein R^{3.1.1} and R^{3.1.2} independently of one another may be H, methyl, ethyl, propyl
   R^{3.2} denotes H or methyl
and pharmacologically acceptable salts, diastereomers, enantiomers, racemates, hydrates or solvates thereof.

Particularly preferred are compounds of formula 1 wherein
R¹ denotes azetidine,
R² denotes piperazine,
R³ denotes O—C₁₋₂-alkylene-phenyl or NH—C₁₋₂-alkylene-phenyl and
R⁴ denotes Cl,
   as well as pharmacologically acceptable salts, diastereomers, enantiomers, racemates, hydrates or solvates thereof.

Preferred compounds of formula 1 are those wherein R², R³ and R⁴ are as hereinbefore defined and wherein R¹ denotes azetidine and pharmacologically acceptable salts, diastereomers, enantiomers, racemates, hydrates or solvates thereof

Preferred compounds of formula 1 are those wherein R¹, R³ and R⁴ are as hereinbefore defined and wherein R² denotes piperazine and pharmacologically acceptable salts, diastereomers, enantiomers, racemates, hydrates or solvates thereof.

Also preferred are compounds of formula 1, wherein R¹, R² and R³ are as hereinbefore defined and wherein R⁴ denotes Cl and pharmacologically acceptable salts, diastereomers, enantiomers, racemates, hydrates or solvates thereof.

Particularly preferred are the novel compounds of formula 1:

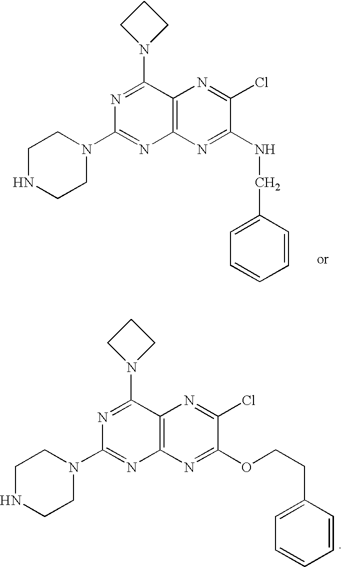

Also preferred are compounds of formula 2

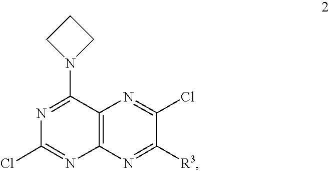

wherein
R³ denotes a group of formula 1a,

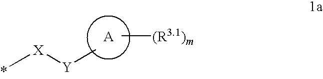

wherein
A denotes a ring selected from among a monocyclic, heterocyclic ring, a bicyclic ring which optionally contains one or more heteroatoms, a C₆₋₁₀-aryl and a C₅₋₁₀-heteroaryl
X denotes NR^{3.2}, O, S;
Y denotes C₁₋₄-alkylene, which may optionally be substituted by one or more R^{3.3},
m denotes 0, 1, 2 or 3
R^{3.1} each independently of one another denote C₁₋₆-alkyl, C₆₋₁₀-aryl, COOR^{3.1.1}, CONR^{3.1.1}R^{3.1.2}, CN, NR^{3.1.1}R^{3.1.2}, NHCOR^{3.1.1}, O—C₁₋₆-haloalkyl, SO₂R^{3.1.1}, SO₂NH₂, halogen, C₁₋₆-haloalkyl, C₁₋₆-alkyl-CONR^{3.1.1}R^{3.1.2}, C₁₋₆-alkyl-NR^{3.1.1}R^{3.1.2}, C₁₋₆-alkyl-CONH₂, O—C₁₋₆-alkylene-NH₂, O—C₃₋₆-cycloalkyl, O—C₁₋₄-alkylene-C₃₋₆-cycloalkyl, O—C₁₋₄-alkylene-CONH₂, SO₂NR^{3.1.1}R^{3.1.2};
   or
R^{3.1} together with two atoms of A forms a 5- or 6-membered carbocyclic ring or a 5- or 6-membered heterocyclic ring, which may optionally contain one or more heteroatoms selected from among oxygen and nitrogen,
wherein
   R^{3.1.1} denotes H, C₁₋₆-alkyl;
   R^{3.1.2} denotes H, C₁₋₆-alkyl;
and
R^{3.2} denotes H, C₁₋₆-alkyl;
and wherein
R^{3.3} each independently of one another denote H, C₁₋₆-alkyl, C₁₋₆-alkyl-OH, C₃₋₆-cycloalkyl, C₃₋₆-cycloalkyl-OH, O—C₁₋₆-alkyl, COOR^{3.1.1}, COO—C₁₋₆-alkyl or CONR^{3.1.1}R^{3.1.2}
or wherein
R^{3.3} together with one or two carbon atoms of Y forms a carbocyclic ring with 3, 4, 5 or 6 carbon atoms,
as well as the pharmacologically acceptable salts, diastereomers, enantiomers, racemates, hydrates or solvates.

In another aspect the invention relates to the above mentioned compounds according to formula 1 for use as medicaments or the use of the above mentioned compounds according to formula 1 for preparing a medicament.

In another preferred aspect the invention relates to the use of compounds according to formula 1 for preparing a medicament for the treatment of diseases that can be treated by inhibiting the PDE4 enzyme.

In particular the present invention relates to the use of the above mentioned compounds according to formula 1 for preparing a medicament for the treatment of respiratory or gastrointestinal complaints or diseases, and also inflammatory diseases of the joints, skin or eyes, cancers, and diseases of the peripheral or central nervous system.

The above mentioned compounds are particularly suitable for preparing a medicament for the prevention and treatment of respiratory or pulmonary diseases associated with increased mucus production, inflammations and/or obstructive diseases of the airways. These include in particular the diseases COPD, chronic sinusitis and asthma.

The above mentioned compounds are also suitable for preparing a medicament for the treatment of inflammatory diseases of the gastrointestinal tract, such as e.g. Crohn's disease and ulcerative colitis.

The above mentioned compounds are also suitable for preparing a medicament for the prevention and/or treatment of diseases of the peripheral or central nervous system such as in particular depression, bipolar or manic depression, acute and chronic anxiety states, schizophrenia, Alzheimer's disease, Parkinson's disease, acute and chronic multiple sclerosis or acute and chronic pain and brain damage caused by stroke, hypoxia or cranio-cerebral trauma.

It is also preferred to use the compounds as defined above to prepare a medicament for the treatment of cancers such as e.g. acute and chronic leukaemias, acute lymphatic leukaemia (ALL) and acute myeloid leukaemia (AML), chronic lymphatic leukaemia (CLL) and chronic myeloid leukaemia (CML), acute non-lymphocytic leukaemia (ANLL), hair cell leukaemia, acute promyelocytic leukaemia (APL), particularly the APL subform with a chromosomal t(15; 17) translocation, diseases of the lymphatic organs, Hodgkin's lymphomas and non-Hodgkin's lymphomas and bone tumours such as e.g. osteosarcoma and all kinds of gliomas such as e.g. oligodendroglioma and glioblastoma.

In the above uses of the pteridine compounds according to the invention for preparing a medicament for the prevention and/or treatment of the above-mentioned diseases as a rule the side effects of the treatment are reduced compared with known therapeutics according to the prior art.

In particular the emesis and nausea that frequently occur as undesirable side effects are reduced when using the compounds according to formula 1.

Terms and Definitions Used

Within the scope of this application, in the definition of possible substituents, these may also be represented in the form of a structural formula. An asterisk (*) in the structural formula of the substituent is to be understood as being the linking point to the rest of the molecule. Thus for example the groups N-piperidinyl (I), 4-piperidinyl (II), 2-tolyl (III), 3-tolyl (IV) and 4-tolyl (V) are shown as follows:

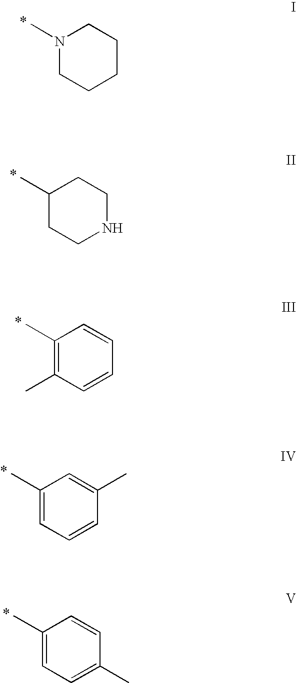

If there is no asterisk (*) in the structural formula of the substituent, each hydrogen atom may be removed at the substituent and the valency thus freed may serve as a binding site to the rest of a molecule. Thus, for example, VI may represent 2-tolyl, 3-tolyl, 4-tolyl and benzyl.

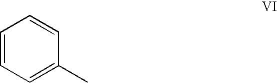

By pharmacologically acceptable acid addition salts are meant for example the salts which are selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate, preferably hydrochloride, hydrobromide, hydrosulphate, hydrophosphate, hydrofumarate and hydromethanesulphonate.

By the term “C₁₋₆-alkyl” (including those which are part of other groups) are meant branched and unbranched alkyl groups with 1 to 6 carbon atoms and by the term “C₁₋₄-alkyl” are meant branched and unbranched alkyl groups with 1 to 4 carbon atoms. Alkyl groups with 1 to 4 carbon atoms are preferred. Examples of these include: methyl, ethyl, n-propyl, iso-propyl, n-butyl, iso-butyl, sec-butyl, tert-butyl, n-pentyl, iso-pentyl, neo-pentyl or hexyl. The abbreviations Me, Et, n-Pr, i-Pr, n-Bu, i-Bu, t-Bu, etc. May also optionally be used for the above-mentioned groups. Unless stated otherwise, the definitions propyl, butyl, pentyl and hexyl include all the possible isomeric forms of the groups in question. Thus, for example, propyl includes n-propyl and iso-propyl, butyl includes iso-butyl, sec-butyl and tert-butyl etc.

By the term “C₁₋₆-alkanol” (including those which are part of other groups) are meant branched and unbranched alkyl groups with 1 to 6 carbon atoms which are substituted by one or more hydroxyl groups and by the term “C₁₋₄-alkanol” are meant branched and unbranched alkyl groups with 1 to 4 carbon atoms which are substituted by one or more hydroxyl groups. C₁₋₆-alkanols which are substituted by a hydroxyl group are also referred to as “monovalent” C₁₋₆-alkanols. C₁₋₆-alkanols which are substituted by two or more hydroxyl groups are also referred to as “polyvalent” C₁₋₆-alkanols. Alkanol groups with 1 to 4 carbon atoms are preferred. Examples of these include: CH₂—OH, ethyl-OH, n-propyl-OH, n-butyl-OH, iso-propyl-OH, n-butyl-OH, iso-butyl-OH, sec-butyl-OH, tert-butyl-OH, n-pentyl-OH, iso-pentyl-OH, neo-pentyl-OH, hexyl-OH,

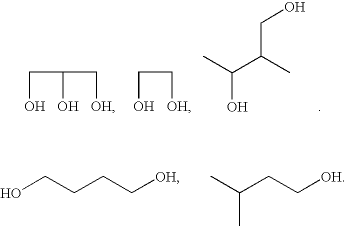

By the term “C₁₋₄-alkylene” (including those which are part of other groups) are meant branched and unbranched alkylene groups with 1 to 4 carbon atoms. Examples of these include: methylene, ethylene, propylene, 1-methylethylene, butylene, 1-methylpropylene, 1,1-dimethylethylene or 1,2-dimethylethylene. Unless stated otherwise, the definitions propylene and butylene include all the possible isomeric forms of the groups in question with the same number of carbons. Thus, for example, propyl also includes 1-methylethylene and butylene includes 1-methylpropylene, 1,1-dimethylethylene, 1,2-dimethylethylene. If the carbon chain is to be substituted by a group which together with one or two carbon atoms of the alkylene chain forms a carbocyclic ring with 3, 4, 5 or 6 carbon atoms, this includes, inter alia, the following examples of the rings:

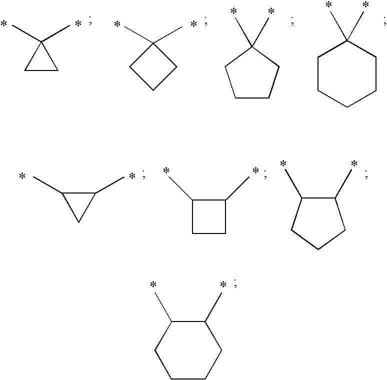

By the term “C₃₋₆-cycloalkyl” (including those which are part of other groups) are meant cyclic alkyl groups with 3 to 6 carbon atoms. Examples of these include: cyclopropyl, cyclobutyl, cyclopentyl or cyclohexyl. Unless otherwise stated, the cyclic alkyl groups may be substituted by one or more groups selected from among methyl, ethyl, iso-propyl, ten-butyl, hydroxy, fluorine, chlorine, bromine and iodine.

By the term “C₇₋₁₁-aralkyl” (including those which are part of other groups) are meant branched and unbranched alkyl groups with 1 to 5 carbon atoms which are substituted by an aromatic ring system with 6 carbon atoms. Examples of these include: benzyl, 1- or 2-phenylethyl. Unless stated otherwise, the aromatic groups may be substituted by one or more groups selected from among methyl, ethyl, iso-propyl, tert-butyl, hydroxy, fluorine, chlorine, bromine and iodine.

“Halogen” within the scope of the present invention denotes fluorine, chlorine, bromine or iodine. Unless stated to the contrary, fluorine, chlorine and bromine are regarded as preferred halogens.

By the term “C₁₋₆-haloalkyl” (including those which are part of other groups) are meant branched and unbranched alkyl groups with 1 to 6 carbon atoms, which are substituted by one or more halogen atoms. By the term “C₁₋₄-alkyl” are meant branched and unbranched alkyl groups with 1 to 4 carbon atoms, which are substituted by one or more halogen atoms. Alkyl groups with 1 to 4 carbon atoms are preferred. Examples of these include: CF₃, CHF₂, CH₂F, CH₂CF₃.

By the term “aryl” or “C₆₋₁₀-aryl” (including those which are part of other groups) are meant aromatic ring systems with 6 or 10 carbon atoms. Examples of these include: phenyl or naphthyl, the preferred aryl group being phenyl. Unless otherwise stated, the aromatic groups may be substituted by one or more groups selected from among methyl, ethyl, iso-propyl, tert-butyl, hydroxy, fluorine, chlorine, bromine and iodine.

By the term “heterocyclic rings”, “heterocyclic group” or “het” are meant five-, six- or seven-membered, saturated or unsaturated heterocyclic rings or 5-10 membered, bicyclic heterorings which may contain one, two or three heteroatoms selected from among oxygen, sulphur and nitrogen, while the ring may be linked to the molecule through a carbon atom or, if available, through a nitrogen atom. The following are examples of five-, six- or seven-membered, saturated or unsaturated heterocyclic rings:

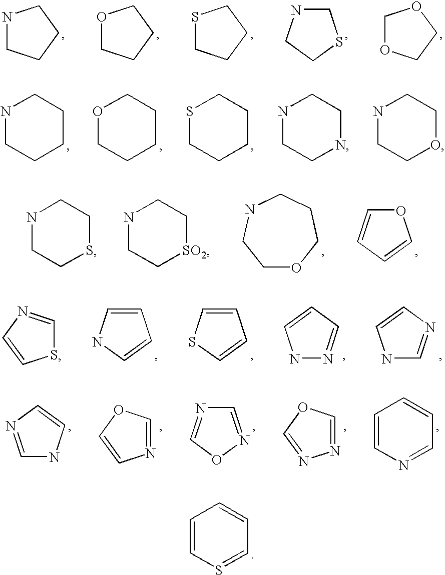

Unless otherwise mentioned, a heterocyclic ring may be provided with a keto group. Examples include.

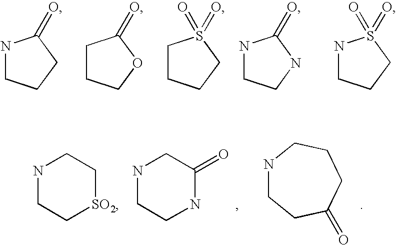

Examples of seven-, eight-, nine- or ten-membered saturated, unsaturated or partially unsaturated bicyclic heterocycles include pyrrolizine, indole, indolizine, isoindole, indazole, purine, quinoline, isoquinoline, benzimidazole, benzofuran, benzopyran, benzothiazole, benzoisothiazole, pyridopyrimidine, pteridine, pyrimidopyrimidine,

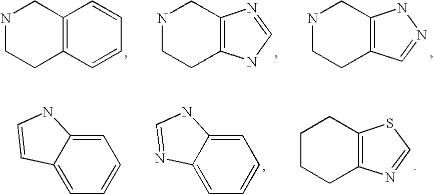

Although encompassed by the term “heterocyclic rings” or “heterocyclic group”, the term “heteroaromatic group” or “heteroaryl” denotes five- or six-membered heterocyclic monocyclic aromatic groups or 5-10 membered, bicyclic heteroaryl rings which may contain one, two or three heteroatoms selected from among oxygen, sulphur and nitrogen, and sufficient conjugated double bonds to form an aromatic system. The ring may be linked to the molecule through a carbon atom or—if available—through a nitrogen atom. Examples of five- or six-membered heteroaryls include:

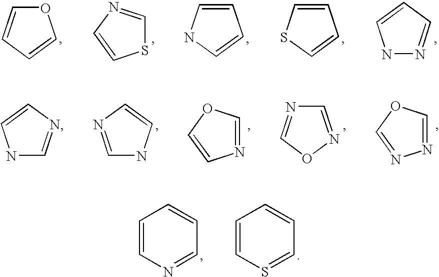

### EXAMPLES

The compounds according to the invention may be prepared by methods known from the literature as described for example in DE 3540952. Other alternative methods of preparing the compounds described below are shown in the following reaction scheme 1 and described in detail in the Examples.

### Example 1

### 4-Azetidin-1-yl-7-benzylamino-6-chloro-2-piperazin-1-yl-pteridine

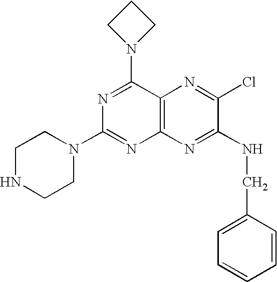

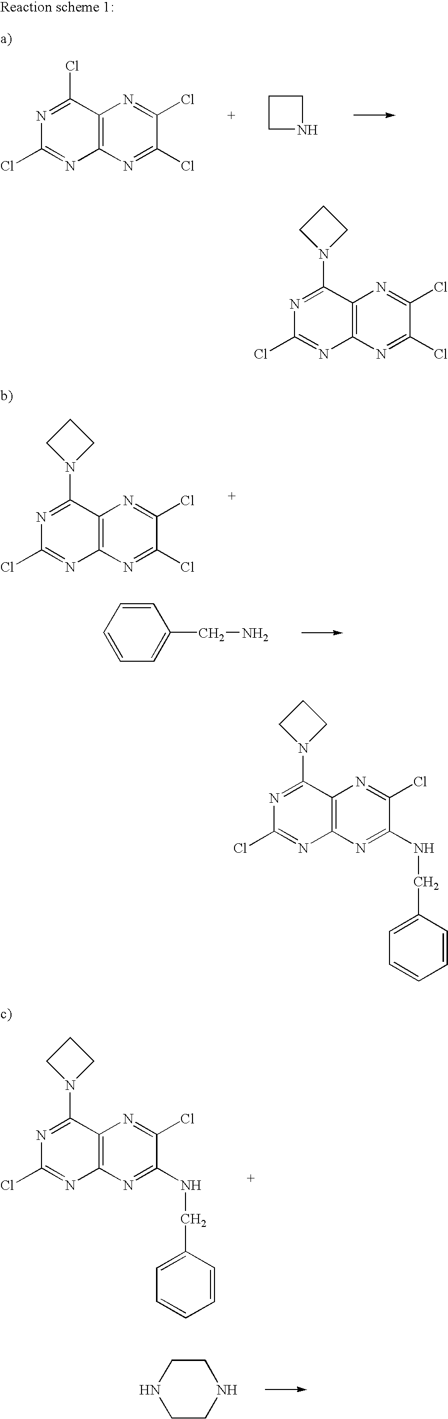

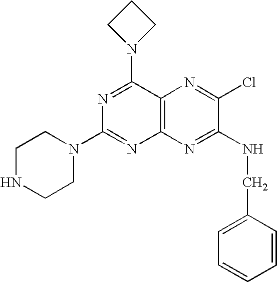

### a) 4-azetidin-1-yl-2,6,7-trichloro-pteridine

2 g (7.4 mmol) tetrachloropteridine are dissolved in approx. 150 ml chloroform and combined with a solution of 1.25 g (14 mmol) sodium hydrogen carbonate in 60 ml of water. The mixture is cooled to 0° C., combined with a solution of 0.5 ml (7.4 mmol) azetidine in approx. 50 ml chloroform and stirred for a further hour at 0° C. Then the organic phase is separated off, washed with water, dried on sodium sulphate and freed from the solvent in vacuo. The residue is triturated with ether and suction filtered. Yield: 780 mg (36% of theoretical), the substance is used in the next step without further purification.

### b) 4-azetidin-1-yl-7-benzylamino-2,6-dichloro-pteridine

740 mg (2 5 mmol) 4-azetidin-1-yl-2,6,7-trichloro-pteridine are dissolved together with 0.45 ml (2.5 mmol) diisopropylethylamine in approx. 30 ml dioxane. To this is added dropwise with stirring a solution of 0.28 ml (2.5 mmol) benzylamine in approx. 5 ml dioxane. After the addition has ended the mixture is stirred for a further 0.5 h. The reaction mixture is poured into 35 ml ice water and stirred, during which time a yellow precipitate is formed which is suction filtered. The precipitate is taken up in dichloromethane and washed with water. The organic phase is dried on sodium sulphate and freed from the solvent in vacuo. The residue is purified by chromatography.

Yield 300 mg (32% of theoretical).

### c) 4-azetidin-1-yl-7-benzylamino-6-chloro-2-piperazin-1-yl-pteridine

300 mg (0.83 mmol) 4-azetidin-1-yl-7-benzylamino-2,6-dichloro-pteridine are suspended in 25 ml dioxane and slowly added dropwise to a solution of 0.36 g (4.2 mmol) piperazine in 20 ml dioxane which has been heated to 80° C. After the addition has ended the mixture is stirred for another 20 min and the reaction mixture is poured into water. It is extracted with dichloromethane, the organic phase is dried on sodium sulphate and freed from the solvent in vacuo. The residue is purified by chromatography.

Yield 80 mg (23% of theoretical) .M+H=411/413

### Example 2

### 4-Azetidin-1-yl-6-chloro-7-(2-phenylethyloxy)-2-piperazin-1-yl-pteridine

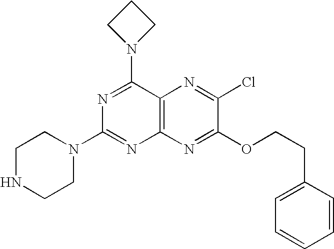

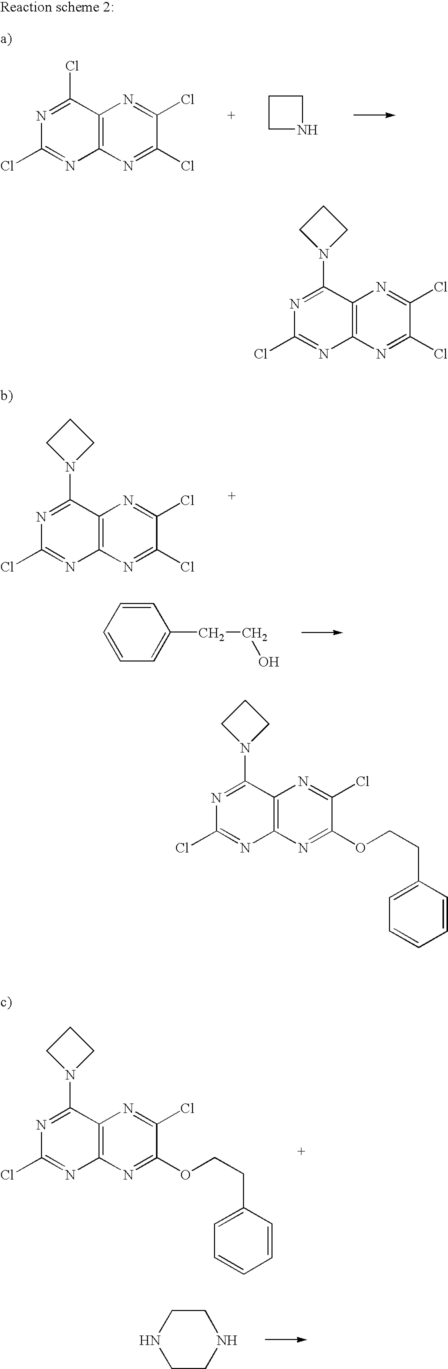

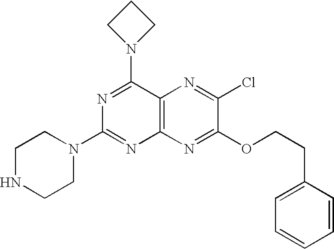

### a) 4-azetidin-1-yl-2,6,7-trichloro-pteridine is Prepared as Described for Example 1

### b) 4-azetidin-1-yl-2,6-dichloro-7-(2-phenylethyloxy)-pteridine

82 μl (0.69 mmol) 2-phenylethanol are dissolved in 5 ml of tetrahydrofuran. The mixture is cooled to approx. −5° C. and 0.35 ml (0 7 mmol) of a 2 molar solution of lithium diisopropylamide in tetrahydrofuran is added with stirring, then the mixture is stirred for 30 min at ambient temperature. It is cooled to −5° C. again and a solution of 200 mg (0.69 mmol) 4-azetidin-1-yl-2,6,7-trichloro-pteridine in 10 ml of tetrahydrofuran is added. The mixture is allowed to come up to ambient temperature and stirred overnight. Approx. 50 ml of water are added and the mixture is extracted with dichloromethane. The organic phase is washed with water, dried on sodium sulphate and freed from the solvent in vacuo. The residue is purified by chromatography. Yield 111 mg (43% of theoretical).

### c) 4-azetidin-1-yl-6-chloro-7-(2-phenylethyloxy)-2-piperazin-1-yl-pteridine

115 mg (1.3 mmol) piperazin are suspended in 2 ml dioxane and heated to 80° C. Then a solution of 100 mg (0.27 mmol) 4-azetidin-1-yl-2,6-dichloro-7-(2-phenethyloxy)-pteridine in 5 ml dioxane is added dropwise, the mixture is stirred for 16 h at 80° C. Then the reaction mixture is freed from the solvent in vacuo, the residue is purified by chromatography. Yield 82 mg (72% of theoretical).

The synthesis of tetrachloropteridine, which is the starting product for the syntheses in reaction schemes 1 and 2, is described in: Schöpf, C.; Reichert, R.; Riefstahl, K. Liebigs Ann. Chem. (1941), 548, 82-94. M+H=426/428

### Indications

As has been found, the compounds of formula 1 are characterised by their wide range of applications in the therapeutic field. Particular mention should be made of those applications for which the compounds according to the invention of formula 1 are preferably suited on account of their pharmaceutical efficacy as PDE4 inhibitors. Examples include respiratory or gastrointestinal diseases or complaints, inflammatory diseases of the joints, skin or eyes, cancers, and also diseases of the peripheral or central nervous system.

Particular mention should be made of the prevention and treatment of diseases of the airways and of the lung which are accompanied by increased mucus production, inflammations and/or obstructive diseases of the airways. Examples include acute, allergic or chronic bronchitis, chronic obstructive bronchitis (COPD), coughing, pulmonary emphysema, allergic or non-allergic rhinitis or sinusitis, chronic rhinitis or sinusitis, asthma, alveolitis, Farmer's disease, hyperreactive airways, infectious bronchitis or pneumonitis, paediatric asthma, bronchiectases, pulmonary fibrosis, ARDS (acute adult respiratory distress syndrome), bronchial oedema, pulmonary oedema, bronchitis, pneumonia or interstitial pneumonia triggered by various causes, such as aspiration, inhalation of toxic gases, or bronchitis, pneumonia or interstitial pneumonia as a result of heart failure, irradiation, chemotherapy, cystic fibrosis (mucoviscidosis), or alphal-antitrypsin deficiency.

Also deserving special mention is the treatment of inflammatory diseases of the gastrointestinal tract. Examples include acute or chronic inflammatory changes in gall bladder inflammation, Crohn's disease, ulcerative colitis, inflammatory pseudopolyps, juvenile polyps, colitis cystica profunda, pneumatosis cystoides intestinales, diseases of the bile duct and gall bladder, e.g. gallstones and conglomerates, for the treatment of inflammatory diseases of the joints such as rheumatoid arthritis or inflammatory diseases of the skin and eyes.

Preferential mention should also be made of the treatment of cancers. Examples include all forms of acute and chronic leukaemias such as acute lymphatic leukaemia (ALL) and acute myeloid leukaemia (AML), acute non-lymphocytic leukaemia (ANLL), chronic lymphatic leukaemia (CLL), chronic myeloid leukaemia (CML), hair cell leukaemia, acute promyelocytic leukaemia (APL), particularly the APL subform with a chromosomal t(15; 17) translocation, diseases of the lymphatic organs, Hodgkin's lymphomas and non-Hodgkin's lymphomas and bone tumours such as e.g. osteosarcoma and all kinds of gliomas such as e.g. oligodendroglioma and glioblastoma.

Preferential mention should also be made of the prevention and treatment of diseases of the peripheral or central nervous system. Examples of these include depression, bipolar or manic depression, acute and chronic anxiety states, schizophrenia, Alzheimer's disease, Parkinson's disease, acute and chronic multiple sclerosis or acute and chronic pain as well as injuries to the brain caused by stroke, hypoxia or craniocerebral trauma.

Particularly preferably the present invention relates to the use of compounds of formula 1 for preparing a pharmaceutical composition for the treatment of inflammatory or obstructive diseases of the upper and lower respiratory tract including the lungs, such as for example allergic rhinitis, chronic rhinitis, bronchiectasis, cystic fibrosis, idiopathic pulmonary fibrosis, fibrosing alveolitis, COPD, chronic bronchitis, chronic sinusitis, asthma, particularly COPD, chronic bronchitis and asthma.

It is most preferable to use the compounds of formula 1 for the treatment of inflammatory and obstructive diseases such as COPD, chronic bronchitis, chronic sinusitis, asthma, Crohn's disease, ulcerative colitis, particularly COPD, chronic bronchitis and asthma.

It is also preferable to use the compounds of formula 1 for the treatment of diseases of the peripheral or central nervous system such as depression, bipolar or manic depression, acute and chronic anxiety states, schizophrenia, Alzheimer's disease, Parkinson's disease, acute and chronic multiple sclerosis or acute and chronic pain as well as injuries to the brain caused by stroke, hypoxia or craniocerebral trauma.

An outstanding aspect of the present invention is the reduced profile of side effects. This means, within the scope of the invention, being able to administer a dose of a pharmaceutical composition without inducing vomiting, preferably nausea and most preferably malaise in the patient. It is particularly preferable to be able to administer a therapeutically effective quantity of substance without inducing emesis or nausea, at every stage of the disease.

### Formulations

In another aspect the invention relates to medicaments for treating respiratory complaints which contain one or more of the above-mentioned pteridines of formula 1.

Suitable forms for administration are for example tablets, capsules, solutions, syrups, emulsions or inhalable powders or aerosols. The content of the pharmaceutically effective compound(s) in each case should be in the range from 0.1 to 90 wt. %, preferably 0.5 to 50 wt. % of the total composition, i.e. in amounts which are sufficient to achieve the dosage range specified hereinafter.

The preparations may be administered orally in the form of a tablet, as a powder, as a powder in a capsule (e.g. a hard gelatine capsule), as a solution or suspension. When administered by inhalation the active substance combination may be given as a powder, as an aqueous or aqueous-ethanolic solution or using a propellant gas formulation.

Preferably, therefore, pharmaceutical formulations are characterised in that they contain one or more compounds of formula 1 according to the preferred embodiments above.

It is particularly preferable if the compounds of formula 1 are administered orally, and it is also particularly preferable if they are administered once or twice a day. Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups containing the active substances or combinations thereof according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. Highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

For oral administration the tablets may, of course, contain, apart from the abovementioned carriers, additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

It is also preferred if the compounds of formula 1 are administered by inhalation, particularly preferably if they are administered once or twice a day. For this purpose, the compounds of formula 1 have to be made available in forms suitable for inhalation. Inhalable preparations include inhalable powders, propellant-containing metered-dose aerosols or propellant-free inhalable solutions, which are optionally present in admixture with conventional physiologically acceptable excipients.

Within the scope of the present invention, the term propellant-free inhalable solutions also includes concentrates or sterile ready-to-use inhalable solutions. The preparations which may be used according to the invention are described in more detail in the next part of the specification.

Inhalable Powders

If the active substances of formula 1 are present in admixture with physiologically acceptable excipients, the following physiologically acceptable excipients may be used to prepare the inhalable powders according to the invention: monosaccharides (e.g. glucose or arabinose), disaccharides (e.g. lactose, saccharose, maltose), oligo- and polysaccharides (e.g. dextran), polyalcohols (e.g. sorbitol, mannitol, xylitol), salts (e.g. sodium chloride, calcium carbonate) or mixtures of these excipients with one another. Preferably, mono- or disaccharides are used, while the use of lactose or glucose is preferred, particularly, but not exclusively, in the form of their hydrates. For the purposes of the invention, lactose is the particularly preferred excipient, while lactose monohydrate is most particularly preferred. Methods of preparing the inhalable powders according to the invention by grinding and micronising and by finally mixing the components together are known from the prior art.

Propellant-Containing Inhalable Aerosols

The propellant-containing inhalable aerosols which may be used according to the invention may contain 1 dissolved in the propellant gas or in dispersed form. The propellant gases which may be used to prepare the inhalation aerosols according to the invention are known from the prior art. Suitable propellant gases are selected from among hydrocarbons such as n-propane, n-butane or isobutane and halohydrocarbons such as preferably fluorinated derivatives of methane, ethane, propane, butane, cyclopropane or cyclobutane. The propellant gases mentioned above may be used on their own or in mixtures thereof. Particularly preferred propellant gases are fluorinated alkane derivatives selected from TG134a (1,1,1,2-tetrafluoroethane), TG227 (1,1,1,2,3,3,3-heptafluoropropane) and mixtures thereof. The propellant-driven inhalation aerosols used within the scope of the use according to the invention may also contain other ingredients such as co-solvents, stabilisers, surfactants, antioxidants, lubricants and pH adjusters. All these ingredients are known in the art.

Propellant-Free Inhalable Solutions

The compounds of formula 1 according to the invention are preferably used to prepare propellant-free inhalable solutions and inhalable suspensions. Solvents used for this purpose include aqueous or alcoholic, preferably ethanolic solutions. The solvent may be water on its own or a mixture of water and ethanol. The solutions or suspensions are adjusted to a pH of 2 to 7, preferably 2 to 5, using suitable acids. The pH may be adjusted using acids selected from inorganic or organic acids. Examples of particularly suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid etc. Preferred inorganic acids are hydrochloric and sulphuric acids. It is also possible to use the acids which have already formed an acid addition salt with one of the active substances. Of the organic acids, ascorbic acid, fumaric acid and citric acid are preferred. If desired, mixtures of the above acids may also be used, particularly in the case of acids which have other properties in addition to their acidifying qualities, e.g. as flavourings, antioxidants or complexing agents, such as citric acid or ascorbic acid, for example. According to the invention, it is particularly preferred to use hydrochloric acid to adjust the pH.

Co-solvents and/or other excipients may be added to the propellant-free inhalable solutions used for the purpose according to the invention. Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavourings, vitamins and/or other additives known in the art. The additives also include pharmacologically acceptable salts such as sodium chloride as isotonic agents. The preferred excipients include antioxidants such as ascorbic acid, for example, provided that it has not already been used to adjust the pH, vitamin A, vitamin E, tocopherols and similar vitamins or provitamins occurring in the human body. Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art.

For the treatment forms described above, ready-to-use packs of a medicament for the treatment of respiratory complaints are provided, containing an enclosed description including for example the words respiratory disease, COPD or asthma, and a pteridine of formula 1.

1. A compound of formula 1, 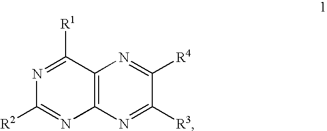
wherein
R¹ denotes a saturated or unsaturated, four-membered heterocyclic group, which contains a nitrogen atom and may optionally contain a further atom selected from nitrogen, sulphur and oxygen;
R² denotes halogen, OR^{2.1}, SR^{2.1}, or NR^{2.1}R^{2.2},
where
R^{2.1} denotes H, C₁₋₄-alkyl, C₆₋₁₀-aryl, or C₇₋₁₁-aralkyl;
R^{2.2} denotes H, C₁₋₄-alkyl, C₆₋₁₀-aryl, or C₇₋₁₁-aralkyl;
or
R² denotes a group selected from C₆₋₁₀-aryl, C₅₋₁₀-heteroaryl and a five-, six- or seven-membered heterocyclic group, which contains a nitrogen atom and which may optionally contain a further atom selected from nitrogen, sulphur and oxygen, while this group may optionally be substituted by a group selected from C₁₋₆-alkyl, O—C₁₋₆-alkyl, C₃₋₆-cycloalkyl, C₇₋₁₁-arallkyl and N(C₁₋₄-alkyl)₂;
R³ denotes a group of formula 1a, 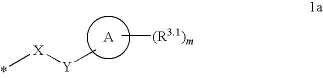
wherein
A denotes a ring selected from a monocyclic, heterocyclic ring, a bicyclic ring which optionally contains one or more heteroatoms, a C₆₋₁₀-aryl and a C₅₋₁₀-heteroaryl;
X denotes NR^{3.2}, O, S;
Y denotes C₁₋₄-alkylene, which may optionally be substituted by one or more R^{3.3};
m denotes 0, 1, 2 or 3;
R^{3.1} each independently of one another denotes C₁₋₆-alkyl, C₆₋₁₀-aryl, COOR^{3.1.1}, CONR^{3.1.1}R^{3.1.2}, CN, NR^{3.1.1}R^{3.1.2}, NHCOR^{3.1.1}, OR^{3.1.1}, O—C₁₋₆-haloalkyl, SO₂R^{3.1.1}, SO₂NH₂, halogen, C₁₋₆-haloalkyl, C₁₋₆-alkyl-CONR^{3.1.1}R^{3.1.2}, C₁₋₆-alkyl-NR^{3.1.1}R^{3.1.2}, C₁₋₆-alkyl-CONH₂, O—C₁₋₆-alkylene-NH₂, O—C₃₋₆-cycloalkyl, O—C₁₋₄-alkylene-C₃₋₆-cycloalkyl, O—C₁₋₄-alkylene-CONH₂, or SO₂NR^{3.1.1}R^{3.1.2};
or
R^{3.1} together with two atoms of A forms a 5- or 6-membered carbocyclic ring or a 5- or 6-membered heterocyclic ring which may optionally contain one or more heteroatoms selected from oxygen and nitrogen,
wherein
R^{3.1.1} denotes H or C₁₋₆-alkyl;
R^{3.1.2} denotes H or C₁₋₆-alkyl;
and
R^{3.2} denotes H or C₁₋₆-alkyl;
and wherein
R^{3.3} each independently of one another denotes H, C₁₋₆-alkyl, C₁₋₆-alkyl-OH, C₃₋₆-cycloalkyl, C₃₋₆-cycloalkyl-OH, O—C₁₋₆-alkyl, COOR^{3.1.1}, COO—C₁₋₆-alkyl, or CONR^{3.1.1}R^{3.1.2}
or
R^{3.3} together with one or two carbon atoms of Y forms a carbocyclic ring with 3, 4, 5 or 6 carbon atoms;
R⁴ denotes a group selected from halogen, C₁₋₆-alkyl, C₂₋₆-alkenyl, C₂₋₆-alkynyl, C₃₋₆-cycloalkyl, C₃₋₆-cycloalkenyl, OR^{4.1}, SR^{4.1}, C₁₋₆-haloalkyl, NR^{4.1}R^{4.2} or a group selected from C₆₋₁₀-aryl , 3-10-membered heterocyclic group and C₅₋₁₀-heteroaryl, which may optionally be substituted by one or more groups selected from C₁₋₆-alkyl, C₁₋₆-haloalkyl, CN, O—C₁₋₆-alkyl, and halogen;
R^{4.1} denotes H, C₁₋₆-alkyl, C₆₋₁₀-aryl, or C₇₋₁₁-arallkyl;
R^{4.2} denotes H, C₁₋₆-alkyl, C₆₋₁₀-aryl, or C₇₋₁₁-arallkyl;
and pharmacologically acceptable salts, diastereomers, enantiomers, racemates, hydrates or solvates thereof

2. The compound of formula 1, according to claim 1, wherein
R¹ denotes an azetidine ring
and pharmacologically acceptable salts, diastereomers, enantiomers, racemates, hydrates or solvates thereof.

3. The compound of formula 1, according to claim 1, wherein
R¹ denotes an azetidine ring,
R² denotes a five- or six-membered heterocyclic group, which contains one or two nitrogen atoms;
and pharmacologically acceptable salts, diastereomers, enantiomers, racemates, hydrates or solvates thereof

4. The compound of formula 1, according to claim 1, wherein
R¹ is an azetidine ring;
R² is a six-membered heterocyclic group which contains two nitrogen atoms;
and pharmacologically acceptable salts, diastereomers, enantiomers, racemates, hydrates or solvates thereof.

5. The compound of formula 1, according to claim 1, wherein
R⁴ denotes a group selected from Cl, F, Br, methyl, ethyl, propyl, 2-methylpropyl, cyclopropyl, cyclohexyl, methoxy, CF₃, NR^{4.1}R^{4.2},
C₆₋₁₀-aryl, optionally substituted by one or more groups selected from methyl, CF₃, CN, methoxy, fluorine, and chlorine,
a five- or six-membered heterocyclic ring, which may contain one or more heteroatoms selected from nitrogen and oxygen,
a five-membered heterocyclic aromatic group, which may contain one or more groups selected from nitrogen and oxygen,
an aromatic or non-aromatic bicyclic group, which may contain one or more heteroatoms selected from sulphur and oxygen;
R^{4.1} denotes H, methyl, or ethyl;
R^{4.2} denotes methyl, ethyl, or phenyl;
and pharmacologically acceptable salts, diastereomers, enantiomers, racemates, hydrates or solvates thereof.

6. The compound of formula 1, according to claim 1, wherein
R⁴ denotes Cl
and pharmacologically acceptable salts, diastereomers, enantiomers, racemates, hydrates or solvates thereof.

7. The compound Gempelinils of formula 1, according to claim 1, wherein
R³ denotes a group of general formula 1a, wherein
A denotes a five-, six- or seven-membered heterocyclic, aromatic or non-aromatic ring, or
an aromatic or non-aromatic, bicyclic ring consisting of eight, nine or ten atoms, which optionally contains one, two or three heteroatoms;
X denotes NR^{3.2}, O, or S;
Y denotes C₁₋₄-alkylene, which may optionally be substituted by one or more R^{3.3};
m denotes 0, 1, 2 or 3;
R^{3.1} each independently of one another denotes C₁₋₄-alkyl, C₆₋₁₀-aryl, COOR^{3.1.1}, CONR^{3.1.1}R^{3.1.2}, CN, NR^{3.1.1}R^{3.1.2}, NHCOR^{3.1.1}, OR^{3.1.1}, O—C₁₋₄-haloalkyl, SO₂R^{3.1.1}, SO₂NH₂, or halogen;
R^{3.1.1} denotes H, or C₁₋₆-alkyl;
R^{3.1.2} denotes H, or C₁₋₆-alkyl;
R^{3.2} denotes H, or C₁₋₆-alkyl;
R^{3.3} each independently of one another denotes C₁₋₆-alkyl, C₁₋₆-alkyl-OH, C₃₋₆-cycloalkyl, O—C₁₋₆-alkyl, COOH, COO—C₁₋₆-alkyl, or CONH₂;
or
R^{3.3} together with one or two carbon atoms of Y forms a carbocyclic ring with 3, 5 or 6 carbon atoms
and pharmacologically acceptable salts, diastereomers, enantiomers, racemates, hydrates or solvates thereof.

8. The compound of formula 1, according to claim 1, wherein
R³ denotes a group of general formula 1a, wherein
A denotes a five-, six- or seven-membered heterocyclic, aromatic or non-aromatic ring, which contains one, two or three heteroatoms, independently of one another, selected from oxygen, nitrogen and sulphur; or
an aromatic or non-aromatic bicyclic ring consisting of eight, nine or ten atoms, which optionally contains one, two or three heteroatoms, independently of one another, selected from oxygen, nitrogen and sulphur;
X denotes NR^{3.2}, O, or S;
Y denotes C₁₋₂-alkylene, which may optionally be substituted by one or more R^{3.3};
m denotes 0, 1, 2 or 3;
R^{3.1} each independently of one another denote C₁₋₄-alkyl, C₆₋₁₀-aryl, COOH, COO—C₁₋₄-alkyl, CONH₂, CN, NH₂, NHCO—C₁₋₄-alkyl, OH, O—C₁₋₄-alkyl, O—C₁₋₄-haloalkyl, SO₂-C₁₋₄-alkyl, SO₂NH₂ , halogen;
R^{3.2} denotes H, C₁₋₄-alkyl;
R^{3.3} denotes H, C₁₋₄-alkyl, C₃₋₆-cycloalkyl, O—C₁₋₄-alkyl, COOH, COO—C₁₋₄-alkyl, or CONH₂,
or
R^{3.3} together with one or two carbon atoms of Y forms a carbocyclic ring with 3, 5 or 6 carbon atoms,
as well as pharmacologically acceptable salts, diastereomers, enantiomers, racemates, hydrates or solvates thereof.

9. The compound of formula 1, according to claim 8, wherein
R^{3.1} each independently of one another denotes methyl, ethyl, propyl, Ph, COOH, COOMe, CONH₂, CN, NH₂, NHCOMe, OH, OMe, OEt, OCF₃, OCHF₂, SO₂Me, SO₂NH₂, F, Cl, or Br;
R^{3.2} denotes H, or C₁₋₄-alkyl;
R^{3.3} denotes H, methyl, ethyl, propyl, butyl, CH₂OH, CH₂CH₂OH, C(CH₂)₂OH, cyclopropyl, COOH, COOMe, COOEt, COOPr, CONH₂, OMe, OEt, or OPr;
or
R^{3.3} together with one or two carbon atoms of Y forms a carbocyclic ring with 3, 5 or 6 carbon atoms
and pharmacologically acceptable salts, diastereomers, enantiomers, racemates, hydrates or solvates thereof

10. The compound of formula 1, according to claim 9, wherein
R^{3.1} denotes methyl, iso-propyl, or tert-butyl;
R^{3.2} denotes H, or methyl;
R^{3.3} denotes H, or methyl;
and pharmacologically acceptable salts, diastereomers, enantiomers, racemates, hydrates or solvates thereof

11. The compound of formula 1 according to claim 1, wherein
R³ denotes a group of general formula 1a, wherein
A denotes a saturated or unsaturated, mono- or bicyclic C₅₋₁₀ heterocycle with 1, 2 or 3 heteroatoms selected from N, O and S;
X denotes NR^{3.2}, or O;
Y denotes C₁₋₂-alkylene, optionally substituted by one or more R^{3.3};
m denotes 0, 1, 2 or 3;
R^{3.1} denotes methyl;
R^{3.2} denotes H;
R^{3.3} denotes H;
and pharmacologically acceptable salts, diastereomers, enantiomers, racemates, hydrates or solvates thereof.

12. The compound of formula 1 according to claim 1, wherein
R³ denotes a group of general formula 1a, wherein
A denotes thiophene, furan, pyrazole, pyridine, isoxazole, thiazole, benzimidazole, benzo[b]thiophene, 2,3-dihydrobenzo[1,4]dioxin, 1,2,3,4-tetrahydronaphthaline, oxazole, tetrahydrofuran or tetrahydropyran;
X denotes NR^{3.2}, or O;
Y denotes C₁₋₂-alkylene, optionally substituted by one or more R^{3.3};
m denotes 0, 1, 2 or 3;
R^{3.1} denotes methyl;
R^{3.2} denotes H;
R^{3.3} denotes H;
and pharmacologically acceptable salts, diastereomers, enantiomers, racemates, hydrates or solvates thereof.

13. The compound of formula 1 according to claim 1, wherein
R¹ denotes azetidine;
R² denotes piperazine;
R³ denotes a group of general formula 1a, wherein
A denotes thiophene, furan, pyrazole, pyridine, isoxazole, thiazole, benzimidazole, benzo[b]thiophene, 2,3-dihydrobenzo[1,4]dioxin, 1,2,3,4-tetrahydronaphthaline, oxazole or phenyl, tetrahydrofuran or tetrahydropyran;
X denotes NR^{3.2}, or O;
Y denotes methylene or ethylene
m denotes 0, 1, 2 or 3;
R^{3.1} each independently of one another denotes methyl, ethyl, phenyl, halogen, COOR^{3.1.1}, CONR^{3.1.1}R^{3.1.2}, CN, NR^{3.1.1}R^{3.1.2}, NHCOR^{3.1.1}, OR^{3.1.1}, O—C₁₋₃-haloalkyl, SO₂R^{3.1.1}, SO₂NH₂, or C₁₋₃-haloalkyl, wherein R^{3.1.1} and R^{3.1.2} independently of one another may be H, methyl, ethyl, or propyl;
R^{3.2} denotes H or methyl;
and pharmacologically acceptable salts, diastereomers, enantiomers, racemates, hydrates or solvates thereof.

14. The compound of formula 1 according to claim 1, wherein
R¹ denotes azetidine;
R² denotes piperazine;
R³ denotes O—C₁₋₂-alkylene-phenyl or NH—C₁₋₂-alkylene-phenyl and
R⁴ denotes Cl;
and pharmacologically acceptable salts, diastereomers, enantiomers, racemates, hydrates or solvates thereof.

15. A compound of formula 2 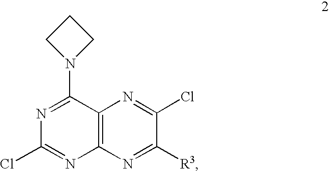
wherein
R³ denotes a group of formula 1a, 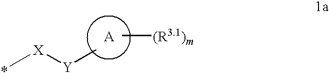
wherein
A denotes a ring selected from among a monocyclic, heterocyclic ring, a bicyclic ring which optionally contains one or more heteroatoms, a C₆₋₁₀-aryl and a C₅₋₁₀-heteroaryt
X denotes NR^{3.2}, O, or S;
Y denotes C₁₋₄-alkylene, which may optionally be substituted by one or more R^{3.3};
m denotes 0, 1, 2 or 3;
R^{3.1} are each independently of one another selected from C₁₋₆-alkyl C₆₋₁₀-aryl, COOR^{3.1.1}, CONR^{3.1.1}R^{3.1.2}, CN, NR^{3.1.1}R^{3.1.2}, NHCOR^{3.1.1}, OR^{3.1.1}, O—C₁₋₆-haloalkyl, SO₂R^{3.1.1}, SO₂NH₂, halogen, C₁₋₆-haloalkyl, C₁₋₆-alkyl-CONR^{3.1.1}R^{3.1.2}, C₁₋₆-alkyl-NR^{3.1.1}R^{3.1.2}, C₁₋₆-alkyl-CONH₂, O—C₁₋₆-alkylene-NH₂, O—C₃₋₆-cycloalkyl, —C₁₋₄-alkylene-C₃₋₆-cycloalkyl, O—C₁₋₄-alkylene-CONH₂ and SO₂NR^{3.1.1}R^{3.1.2};
or
wherein
R^{3.1} together with two atoms of A forms a 5- or 6-membered carbocyclic ring or a 5- or 6-membered heterocyclic ring, which may optionally contain one or more heteroatoms selected from oxygen and nitrogen,
wherein
R^{3.1.1} denotes H, or C₁₋₆-alkyl;
R^{3.1.2} denotes H, or C₁₋₆-alkyl;
and
R^{3.2} denotes H, or C₁₋₆-alkyl;
and wherein
R^{3.3} each independently of one another denote H, C₁₋₆-alkyl, C₁₋₆-alkyl-OH, C₃₋₆-cycloalkyl, C₃₋₆-cycloalkyl-OH, O—C₁₋₆-alkyl, COOR^{3.1.1}, COO—C₁₋₆-alkyl or CONR^{3.1.1}R^{3.1.2}
or wherein
R^{3.3} together with one or two carbon atoms of Y forms a carbocyclic ring with 3, 4, 5 or 6 carbon atoms,
as well as the pharmacologically acceptable salts, diastereomers, enantiomers, racemates, hydrates or solvates.

16. (canceled)

17. A method for treating diseases associated with an inhibition of PDE4 enzyme comprising administering to a patient in need thereof a compound according to claim 1.

18. A method for treating respiratory or gastrointestinal complaints, or inflammatory diseases of the joints, skin or eyes, cancers, or diseases of the peripheral or central nervous system, comprising administering to a patient in need thereof a compound according to claim 1.

19. A method for preventing or treating respiratory or pulmonary diseases which are accompanied by increased mucus production, inflammations and/or obstructive diseases of the respiratory tract comprising administering to a patient in need thereof a compound according to claim 1.

20. A method for treating inflammatory diseases of the gastrointestinal tract comprising administering to a patient in need thereof a compound according to claim 1.

21. A method for treating inflammatory and obstructive diseases comprising administering to a patient in need thereof a compound according to claim 1, wherein the inflammatory and obstructive disease is COPD, chronic sinusitis, asthma, Crohn's disease or ulcerative colitis.

22. A method for preventing or treating diseases of the peripheral or central nervous system comprising administering to a patient in need thereof a compound according to claim 1, wherein the peripheral or central nervous system disease is depression, bipolar or manic depression, acute or chronic anxiety states, schizophrenia, Alzheimer's disease, Parkinson's disease, acute or chronic multiple sclerosis, acute pain, chronic pain or brain damage caused by stroke, hypoxia or cranio-cerebral trauma.

23. A method for treating cancers comprising administering to a patient in need thereof a compound according to claim 1, wherein the cancer is acute or chronic leukaemia, acute lymphatic or acute myeloid leukaemia, chronic lymphatic or chronic myeloid leukaemia, diseases of the lymphatic organs, Hodgkin's lymphomas, non-Hodgkin's lymphomas, bone tumor or gliomas.

24. The method according to anyone of claims 17-23, wherein side effects of the treatment are reduced and wherein the reduced side effect is emesis, nausea or combination thereof.

25. (canceled)

26. The method according to claim 23, wherein the bone tumor is osteosarcoma.

27. The method according to claim 23, wherein the glioma is oligodendroglioma or glioblastoma.
